(19) United States
(12) Patent Application Publication
(10) Pub. No.: US 2026/0265371 A1
(43) Pub. Date: Sep. 10, 2026

(54) CHIMERIC ANTIGEN RECEPTOR TARGETING BCMA AND USE THEREOF

(71) Applicants: NATIONAL CANCER CENTER, Goyang-si (KR); PROTANBIO INC., Seoul (KR)

(72) Inventors: Sangjin LEE, Paju-si (KR); Hyeonseok EOM, Seoul (KR); Eunjung PARK, Seoul (KR); Bumkyu CHOI, Paju-si (KR); Soyoun RO, Incheon (KR); Seongwon HONG, Goyang-si (KR); Chungyong HAN, Goyang-si (KR); Jeyoel CHO, Seoul (KR); Hyejin SUNG, Seoul (KR); Siyoung CHOI, Seoul (KR)

(73) Assignees: NATIONAL CANCER CENTER, Seoul (KR); PROTANBIO INC., Goyang-si (KR)

(21) Appl. No.: 19/683,631

(22) Filed: May 20, 2026

Related U.S. Application Data
(63) Continuation of application No. 18/002,109, filed on May 28, 2021, now Pat. No. 12,649,785.

(30) Foreign Application Priority Data
Jun. 17, 2020 (KR) 10-2020-0073714

(51) Int. Cl. C07K 16/28; A61K 40/11; A61K 40/31; A61K 40/42; C12N 15/86

(52) U.S. Cl. CPC C07K 16/28; A61K 40/11; A61K 40/31; A61K 40/4215; C12N 15/86; C07K 2317/53; C07K 2317/565; C12N 2740/15032; C12N 2740/15041

(57) ABSTRACT

The present invention relates to a chimeric antigen receptor targeting BCMA and uses thereof, and more particularly, to an antibody targeting BCMA, a chimeric antigen receptor comprising the antibody, and CAR-T cells expressing the chimeric antigen receptor.

It was confirmed that the chimeric antigen receptor and the CAR-T cell targeting BCMA prepared in the present invention specifically bound to the antigen (BCMA), and activation of the BCMA-bound CAR-T cells was made.

In addition, since it was confirmed that the CAR-T cell of the present invention effectively killed BCMA-expressing cells, the chimeric antigen receptor and the CAR-T cell targeting BCMA of the present invention can be applied for use in preventing or treating diseases related to B cell expression or BCMA expression.

### CROSS-REFERENCE TO RELATED APPLICATIONS

This application is a continuation of U.S. patent application Ser. No. 18/002,109, which has a 35 U.S.C. 371(c) date of Dec. 16, 2022, and is the United States national stage of International Patent Appl. No. PCT/KR2021/006671, filed May 28, 2021, which claims priority to Korean Patent Appl. No. 10-2020-0073714, filed Jun. 17, 2020. Each of these applications is hereby incorporated herein.

### SEQUENCE LISTING

A sequence listing is submitted electronically herewith. The sequence listing is named 562648US_Sequence Listing_ST26, was created May 20, 2026, and is 48,563 bytes in size. The sequence listing is hereby incorporated herein.

### TECHNICAL FIELD

The present invention relates to a chimeric antigen receptor targeting BCMA and uses thereof, and more particularly, to an antibody targeting BCMA, a chimeric antigen receptor comprising the antibody, and a CAR-T cell expressing the chimeric antigen receptor.

### BACKGROUND ART

Chimeric antigen receptor (CAR) T-cells are molecules that combine antibody-based specificity for a desired antigen with a T cell receptor-activating intracellular domain to generate chimeric proteins that exhibit specific anti-cancer immune activity. In general, a chimeric antigen receptor (CAR) comprises an extracellular antigen binding domain, a transmembrane domain and an intracellular signaling domain. The extracellular antigen binding domain can comprise a single-chain variable fragment (scFv) targeting the identified tumor antigen.

CARs can be expressed on the surface of T cells, which are immune effector cells, using genetic transfection techniques. Upon binding of a CAR expressed on the surface of a T cell to a target tumor antigen, the CAR can activate the T cell to initiate a specific antitumor response in an antigen-dependent manner.

Meanwhile, B-cell maturation antigen (BCMA), also known as CD269 or TNFRSF17, is a member of the tumor necrosis factor receptor family. It has been reported that BCMA can bind to B-cell activating factor receptor (BAFF) and B-cell proliferation-inducing ligand (APRIL) to promote survival of B cells at different developmental stages. Abnormal signaling can lead to abnormal proliferation of B cells, leading to autoimmune diseases such as multiple myeloma and tumorigenesis (Rickert, et al., Immunological Reviews, 2011, Vol. 244:115-133).

Multiple myeloma is a type of blood cancer that occurs when plasma cells are abnormally differentiated and proliferated, which creates tumors and melts bones, causing pain. In addition, multiple myeloma invades the bone marrow and decreases white blood cell, red blood cell, and platelet counts, increasing the risk of anemia, infection, and bleeding. Furthermore, myeloma cells also produce M protein, which is an abnormal immune protein, which increases the concentration of blood, causing blood hyperviscosity syndrome or damage to the kidneys.

Some treatments for multiple myeloma are similar to treatments for other cancers, such as chemotherapy or radiation therapy, stem cell transplantation or bone marrow transplantation, targeted therapy or biological therapy. Antibody-based cell immunotherapy has shown practical clinical benefit for patients with hematologic malignancies, particularly B-cell non-Hodgkin's lymphoma, but most patients have problems with relapse or secondary resistance, so an effective immunotherapeutic agent for treating multiple myeloma is required. For this purpose, a treatment method using CAR-T cells is being studied (Ellebrecht et al., Science 353:179-184, 2016; Carpenter et al., Clin Cancer Res, 19(8): 2048-2060, 2013; WO2016-014789; WO 2016/014565; WO 2013/154760).

### DISCLOSURE

### Technical Problem

In the present invention, as a result of earnest efforts to develop a therapeutic agent for diseases related to B cells such as multiple myeloma, an antibody targeting BCMA was selected, and the selected anti-BCMA antibody was humanized to finally obtain a BCMA-targeting chimeric antigen receptor and a CAR-T cell were prepared. It was confirmed that the present BCMA-targeting CAR-T cell bound to BCMA and effectively killed BCMA-expressing tumor cells.

Accordingly, it is one of the objects of the present invention to provide an antibody targeting BCMA and a chimeric antigen receptor comprising the antibody.

Another object of the present invention is to provide a polynucleotide encoding a chimeric antigen receptor targeting BCMA, a vector comprising the same, and immune effector cells expressing the chimeric antigen receptor comprising the polynucleotide or the vector.

Another object of the present invention is to provide a pharmaceutical composition for preventing or treating a disease related to BCMA expression, including immune effector cells expressing the BCMA-targeting chimeric antigen receptor.

### Technical Solution

In order to achieve the above object, the present invention can provide an antibody specifically binding to BCMA (B-cell maturation antigen) or fragment thereof, comprising: a heavy chain variable region including a CDR1 region represented by the amino acid sequence of SEQ ID NO: 1, a CDR2 region represented by the amino acid sequence of SEQ ID NO: 2, and a CDR3 region represented by the amino acid sequence of SEQ ID NO: 3; and a light chain variable region including a CDR1 region represented by the amino acid sequence of SEQ ID NO: 4, a CDR2 region represented by the amino acid sequence of SEQ ID NO: 5, and a CDR3 region represented by the amino acid sequence of SEQ ID NO: 6.

In a preferred embodiment of the present invention, the antibody can comprise a heavy chain variable region represented by the amino acid sequence of SEQ ID NO: 7 and a light chain variable region represented by the amino acid sequence of SEQ ID NO: 8; or a heavy chain variable region represented by the amino acid sequence of SEQ ID NO: 13 and a light chain variable region represented by the amino acid sequence of SEQ ID NO: 14.

In addition, the present invention can provide a chimeric antigen receptor (CAR) comprising: a BCMA-binding domain; a transmembrane domain; a costimulatory domain; and an intracellular signal transduction domain,
wherein the BCMA-binding domain has an antibody specifically binding to BCMA or fragment thereof comprising: a heavy chain variable region comprising a CDR1 region represented by the amino acid sequence of SEQ ID NO: 1, a CDR2 region represented by the amino acid sequence of SEQ ID NO: 2 and a CDR3 region represented by the amino acid sequence of SEQ ID NO: 3; and a light chain variable region comprising a CDR1 region represented by the amino acid sequence of SEQ ID NO: 4, a CDR2 region represented by the amino acid sequence of SEQ ID NO: 5, and a CDR3 region represented by the amino acid sequence of SEQ ID NO: 6.

In a preferred embodiment of the present invention, the transmembrane domain may be derived from a protein selected from the group consisting of CD8a, CD4, CD28, CD137, CD80, CD86, CD152 and PD1.

In another preferred embodiment of the present invention, the costimulatory domain may be derived from a protein selected from the group consisting of CD28, 4-1BB, OX-40 and ICOS, and the signaling domain may be derived from CD3ζ.

In another preferred embodiment of the present invention, it may further include a hinge region located between the C-terminus of the BCMA-binding domain and the N-terminus of the transmembrane domain, wherein the hinge region is CD8a may be of origin.

To achieve another object, the present invention provides a polynucleotide encoding the chimeric antigen receptor (CAR).

The present invention also provides a vector comprising a polynucleotide encoding a chimeric antigen receptor (CAR).

In a preferred embodiment of the present invention, the vector may be a plasmid, a retroviral vector or a lentiviral vector.

In addition, the present invention provides an immune effector cell comprising a polynucleotide encoding the chimeric antigen receptor (CAR) or the vector comprising the polynucleotide encoding the chimeric antigen receptor (CAR).

In a preferred embodiment of the present invention, the immune effector cells may be T cells.

In order to achieve another object, the present invention provides a pharmaceutical for preventing or treating a disease relating to BCMA expression comprising an immune effector cell expressing the chimeric antigen receptor targeting BCMA or an antibody or fragment thereof that specifically binds to BCMA.

In a preferred embodiment of the present invention, the disease relating to BCMA expression may be multiple myeloma, hematologic cancer, non-Hodgkin's lymphoma, autoantibody-dependent autoimmune disease, systemic lupus erythematosus (SLE) or rheumatoid arthritis.

### Advantageous Effects

It was confirmed that the chimeric antigen receptor and CAR-T cells targeting BCMA prepared in the present invention specifically bound to the antigen, BCMA, and activation of the BCMA-bound CAR-T cells was made.

In addition, since it was confirmed that the CAR-T cells of the present invention effectively killed BCMA-expressing cells, the chimeric antigen receptor and CAR-T cells targeting BCMA of the present invention can be usefully used for preventing or treating diseases related to B cell expression or BCMA expression.

### DESCRIPTION OF DRAWINGS

FIG. 1 is data confirming the BCMA binding ability of a humanized anti-BCMA monoclonal antibody (3G4V2).

FIG. 2 is a schematic diagram illustrating the lentiviral vector expressing a chimeric antigen receptor (hBCMA-CAR) targeting BCMA and a chimeric antigen receptor expressed in T cells.

FIG. 3 is a schematic diagram illustrating a method for preparing hBCMA-CAR-T cells using the lentivirus expressing hBCMA-CAR.

FIG. 4 is data confirming the BCMA binding ability of hBCMA-CAR-T cells.

FIG. 5 is data confirming the expression level of IFNγ and CD107a by 3G4V2-CAR T cells in the presence of target cells in order to confirm the activation of hBCMA-CAR-T cells.

FIG. 6 is data confirming the killing effect of target cells by hBCMA-CAR-T cells.

### MODE OF THE INVENTION

Hereinafter, the present invention will be described in detail.

### Antibodies Targeting BCMA

In one aspect, the present invention relates to an antibody specifically binding to BCMA (B-cell maturation antigen) or fragment thereof comprising: a heavy chain variable region including a CDR1 region represented by the amino acid sequence of SEQ ID NO: 1, a CDR2 region represented by the amino acid sequence of SEQ ID NO: 2, and a CDR3 region represented by the amino acid sequence of SEQ ID NO: 3; and a light chain variable region including a CDR1 region represented by the amino acid sequence of SEQ ID NO: 4, a CDR2 region represented by the amino acid sequence of SEQ ID NO: 5, and a CDR3 region represented by the amino acid sequence of SEQ ID NO: 6.

In the present invention, the antibody may be a monoclonal antibody. In the present invention, the term “monoclonal antibody” is an antibody produced by a single antibody-forming cell, and has a uniform primary structure (amino acid sequence). It recognizes only one antigenic determinant and is generally produced by culturing a hybridoma cell in which cancer cells and antibody-producing cells are fused. It can also be produced by using other recombinant protein expression host cells using the obtained antibody gene sequence.

In the present invention, the term “antibody” can be used not only in a complete form having two full-length light chains and two full-length heavy chains, but also as a fragment of an antibody molecule. A fragment of an antibody molecule refers to a fragment having at least a peptide tag (epitope) binding function, and includes scFv, Fab, F(ab′), F(ab′)₂, a single domain, and the like.

Among the antibody fragments, Fab has a structure having variable regions of light and heavy chains, a constant region of a light chain and a first constant region (CH1) of a heavy chain, and has one antigen-binding site. Fab′ differs from Fab in that it has a hinge region comprising one or more cysteine residues at the C terminus of the heavy chain CH1 domain. The F(ab′) 2 antibody is produced by forming a disulfide bond with a cysteine residue in the hinge region of Fab′. Fv is a minimal antibody fragment having only a heavy chain variable region and a light chain variable region. Recombinant technology for generating Fv fragments is described in International Patent Publication Nos. WO 88/10649, WO 88/106630, WO 88/07085, WO 88/07086 and WO 88/09344. Double-chain Fv (dsFv) has a heavy chain variable region and a light chain variable region connected by a disulfide bond, and in single-chain Fv (scFv), the variable region of the heavy chain and the variable region of the light chain are generally covalently linked through a peptide linker. Such antibody fragments can be obtained using proteolytic enzymes (e.g., papain-restricted digestion of the entire antibody yields Fab, and pepsin digestion yields F(ab′)₂ fragments). Preferably, it can be produced through genetic recombination technology.

In the present invention, the present monoclonal antibody specifically binding to BCMA can be prepared by using all or part of BCMA protein/peptide as an immunogen (or antigen). More specifically, first, as an immunogen, BCMA, a fusion protein comprising the BCMA protein, or a carrier comprising the BCMA protein, if necessary, together with an adjuvant (e.g., Freund adjuvant), is injected once or more by subcutaneous, intramuscular, intravenous, intraperitoneal in mammals except for humans to achieve an immunization. The mammals other than humans are preferably mice, rats, hamsters, malmots, chickens, rabbits, cats, dogs, pigs, goats, sheep, donkeys, horses or cattle (including transgenic animals engineered to produce an antibody from other animals such as mice to produce human antibody), more preferably mouse, rat, hamster, malmot, chicken or rabbit. Antibody-producing cells can be obtained from the immune-sensitized mammal about 1 to 10 days after the final immunization by performing immunization 1 to 4 times every 1 to 21 days from the first immunization. The number of times and intervals for immunization can be appropriately changed depending on the characteristics of the immunogen to be used.

Preparation of a hybridoma secreting a monoclonal antibody can be carried out according to the method of Keira and Mirstein et al. (Nature, 1975, Vol. 256, p. 495-497) and a method similar thereto. Hybridomas can be produced by cell fusion of mammal-derived myeloma cells without autologous antibody-producing ability and antibody-producing cells contained in the group consisting of spleen, lymph node, bone marrow and tonsils, preferably spleen.

For cell fusion, for example, a fusion promoter including polyethylene glycol or Sendai virus or a method by electric pulse is used, for example, antibody-producing cells and mammalian-derived cells capable of indefinite proliferation are suspended at a ratio of about 1:1 to 1:10 in a fusion medium containing a fusion promoter, and in this state, cultured at about 30 to 40° C. for about 1 to 5 minutes. As the fusion medium, for example, MEM medium, RPMI1640 medium, and Iscove's Modified Dulbecco's Medium may be used, and it is preferable to exclude serums such as bovine serum.

In the method of screening the hybridoma clones producing the monoclonal antibody, first, the fusion cells obtained as described above are transferred to a selection medium such as HAT medium, and cultured at about 30 to 40° C. for about 3 days to 3 weeks to kill cells other than hybridomas. Then, after culturing the hybridoma on a microtiter plate, etc., the part with increased reactivity between the immunogen used for the immune response of animals other than humans described above and the culture supernatant was subjected to RIA (radioactive substance-marked immuno antibody) or ELISA (Enzyme-Linked Immunosorbent Assay). The clone producing the monoclonal antibody found above shows specific binding ability to the immunogen.

The monoclonal antibody of the present invention can be obtained by culturing such a hybridoma in vitro or in vivo. For culturing, a conventional method for culturing cells derived from mammals is used, and for collecting monoclonal antibody from a culture or the like, a conventional method in this field for purifying an antibody in general is used. As each method, for example, salting out, dialysis, filtration, concentration, centrifugation, fractional precipitation, gel filtration chromatography, ion exchange chromatography, affinity chromatography, high-performance liquid chromatography, gel electrophoresis or isoelectric point electrophoresis, etc. can be applied, and these are applied in combination as needed. The purified monoclonal antibody is then concentrated and dried to be in a liquid or solid state depending on the use.

In addition, the monoclonal antibody of the present invention can be obtained by: synthesized each ligated gene of DNAs encoding heavy chain and light chain variable regions, respectively, with known DNA encoding the constant regions of heavy chain and light chain (e.g., Japan patent publication No. 2007-252372) by PCR or chemical synthesis; transplanted into a known expression vector that enables expression of the gene, etc. to prepare a transformant; producing an antibody by expressing it in a host of CHO cells, Escherichia coli, etc.; and purifying the antibody from this culture solution using a protein A or G column or the like.

In a specific embodiment of the present invention, a mouse antibody that specifically binds to BCMA was prepared and screened to establish a novel antibody, which was named 3G4.

In the present invention, it was confirmed that the present 3G4 antibody comprised a heavy chain variable region having a CDR1 region represented by the amino acid sequence of SEQ ID NO: 1 (GYTFTSYV), a CDR2 region represented by the amino acid sequence of SEQ ID NO: 2 (IIPYNDGT), and a CDR3 region represented by the amino acid sequence of SEQ ID NO: 3 (ARWNWDGYFDV); and a light chain variable having a CDR1 region represented by the amino acid sequence of SEQ ID NO: 4 (KSLLHSNGITY), a CDR2 region represented by the amino acid sequence of SEQ ID NO: 5 (QMS), and a CDR3 region represented by the amino acid sequence of SEQ ID NO: 6 (TONLELPFT).

Preferably, the 3G4 antibody can be composed of a heavy chain variable region represented by the amino acid sequence of SEQ ID NO: 7 and a light chain variable region represented by the amino acid sequence of SEQ ID NO: 8. In addition, the heavy chain variable region of the 3G4 antibody may be encoded by the nucleotide sequence of SEQ ID NO: 9, and the light chain variable region of the 3G4 antibody may be encoded by the nucleotide sequence of SEQ ID NO: 10. When the 3G4 antibody exists in scFv form, it may be linked to a light chain variable region-linker-heavy chain variable region, and preferably encoded by the amino acid sequence of SEQ ID NO: 11 or the nucleotide sequence of SEQ ID NO: 12.

In another specific embodiment of the present invention, a humanized antibody obtained by changing the anti-BCMA antibody 3G4 into a structure corresponding to a human was prepared, which was named 3G4V2.

The heavy chain variable region CDRs and light chain variable region CDRs of 3G4V2 are the same as those of 3G4, and the remaining parts except for the CDR part were humanized. Preferably, 3G4V2 can be composed of a heavy chain variable region represented by the amino acid sequence of SEQ ID NO: 13 and a light chain variable region represented by the amino acid sequence of SEQ ID NO: 14. In addition, the heavy chain variable region of the 3G4V2 antibody may be encoded by the nucleotide sequence of SEQ ID NO: 15, and the light chain variable region of the 3G4V2 antibody may be encoded by the nucleotide sequence of SEQ ID NO: 16. When the 3G4 antibody exists in scFv form, it may be linked to a light chain variable region-linker-heavy chain variable region, and preferably encoded by the amino acid sequence of SEQ ID NO: 17 or the nucleotide sequence of SEQ ID NO: 18.

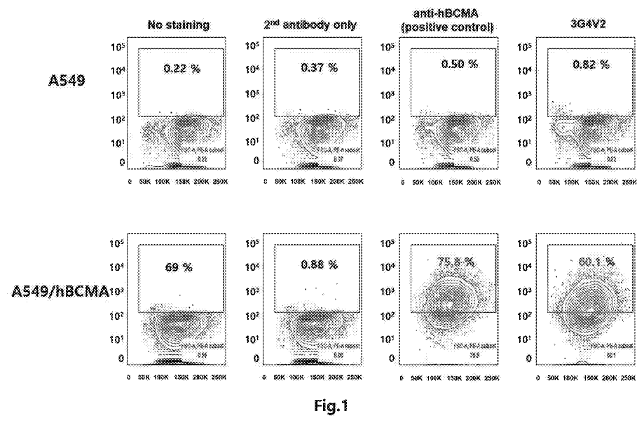

In another specific embodiment of the present invention, as a result of confirming whether the humanized anti-BCMA antibody 3G4V2 and BCMA specifically bind, as shown in FIG. 1, binding to 3G4V2 was not confirmed in A549 cells in which hBCMA is not expressed. However, it was confirmed that the binding capacity of A549 cells expressing hBCMA and 3G4V2 was increased.

Therefore, in the present invention, a chimeric antigen receptor (CAR) targeting BCMA was prepared using a humanized anti-BCMA antibody, 3G4V2.

As used herein, the term “humanized antibody” is an antibody that possesses an amino acid sequence corresponding to that of an antibody produced by a human and/or is made using one of the techniques for making human antibodies as disclosed herein. This definition of a humanized antibody specifically excludes a humanized antibody comprising non-human antigen-binding residues.

In addition, it should be understood that the protein, polypeptide and/or amino acid sequence encompassed by the present invention includes functional variants or homologues having at least the same or similar function as the protein or polypeptide.

In the present invention, functional variants may be a protein or polypeptide obtained by substituting, deleting or adding one or more amino acids in the amino acid sequence of the protein and/or polypeptide. Functional variants are capable of substantially retaining the biological properties of an unmodified protein or polypeptide. For example, functional variants may retain at least 60%, 70%, 80%, 90% or 100% of the biological activity (such as antigen binding capacity) of the original protein or polypeptide.

In the present invention, homologues may be a protein or polypeptide having at least about 85% (e.g., about 85%, 90%, 91%, 92%, 93%, 94%, 95%, 96%, 97%, 98%, 99% or more) amino acid sequence homology with the protein and/or polypeptide (e.g., an antibody capable of specifically binding BCMA or a fragment thereof).

In the present invention, homology generally refers to similarity, similarity or correlation between two or more sequences.

### Chimeric Antigen Receptor Targeting BCMA

The present invention from another point of view, also relates to a chimeric antigen receptor (CAR) comprising: a BCMA-binding domain; a transmembrane domain; a costimulatory domain; and an intracellular signal transduction domain, wherein the BCMA-binding domain includes an antibody or fragment thereof capable of specifically binding BCMA comprising: a heavy chain variable region comprising a CDR1 region represented by the amino acid sequence of SEQ ID NO: 1, a CDR2 region represented by the amino acid sequence of SEQ ID NO: 2 and a CDR3 region represented by the amino acid sequence of SEQ ID NO: 3, and a light chain variable region comprising a CDR1 region represented by the amino acid sequence of SEQ ID NO: 4, a CDR2 region represented by the amino acid sequence of SEQ ID NO: 5, and a CDR3 region represented by the amino acid sequence of SEQ ID NO: 6.

As used herein, the term “chimeric antigen receptor (CAR)” generally refers to a fusion protein containing an extracellular domain having the ability to bind an antigen and one or more intracellular domains. A CAR is a core part of a chimeric antigen receptor T cell (CAR-T) and may contain an antigen binding domain, a transmembrane domain, a co-stimulatory domain, and an intracellular signal transduction domain. A CAR can be combined with a T cell receptor-activating intracellular domain based on the antigen (e.g., BCMA) specificity of the antibody. Genetically modified CAR-expressing T cells can specifically identify and eliminate target antigen-expressing malignant cells.

In the present invention, the term “BCMA” refers to a B-cell maturation antigen. BCMA (also known as TNFRSF17, BCM or CD269) is a member of the tumor necrosis receptor (TNFR) family and is predominantly expressed on terminally differentiated B cells, such as memory B cells and plasma cells. BCMA is expressed on tumor cells (e.g., multiple myeloma cells) or localized on the surface of tumor cells. “BCMA” of the present invention may include proteins comprising mutations of full-length wild-type BCMA, e.g., point mutations, fragments, insertions, deletions and splice variants.

In the present invention, the term “BCMA-binding domain” generally refers to a domain capable of specifically binding to a BCMA protein. For example, the BCMA-binding domain may contain an anti-BCMA antibody or fragment thereof capable of specifically binding to a human BCMA polypeptide or fragment thereof expressed in a B cell.

In the present invention, the term “binding domain” can be used interchangeably refers to “extracellular domain”, “extracellular binding domain”, “antigen-specific binding domain” and “extracellular antigen-specific bidding domain” and refers to a CAR domain or fragment having the ability to specifically bind a target antigen (e.g., BCMA).

In the present invention, the anti-BCMA antibody or fragment thereof is the above-described anti-BCMA antibody, a monoclonal antibody, preferably a single chain variable fragment (scFv), and in the present invention, a humanized anti-BCMA antibody, 3G4V2 was used.

In the present invention, the term “transmembrane domain” generally refers to a domain of a CAR that passes through a cell membrane and is connected to an intracellular signaling domain to play a signaling role. The transmembrane domain may be derived from a protein selected from the group consisting of CD8a, CD4, CD28, CD137, CD80, CD86, CD152 and PD1, and may preferably be represented by the amino acid sequence of SEQ ID NO: 29.

In the present invention, the term “costimulatory domain” generally refers to an intracellular domain capable of providing immune-stimulatory molecules, which are cell surface molecules required for an effective response of lymphocytes to antigens. The costimulatory domain described above may comprise a costimulatory domain of CD28, and may comprise a costimulatory domain of the TNF receptor family, such as the costimulatory domain of OX40 and 4-1BB, and preferably it may be 4-1BB represented by the amino acid sequence of SEQ ID NO: 30.

In the present invention, the term “intracellular signal transduction domain” generally refers to a domain located inside a cell and capable of transmitting a signal. In the present invention, the intracellular signaling domain is the intracellular signaling domain of the chimeric antigen receptor. For example, the intracellular signaling domain may be selected from CD3ζ intracellular domain, CD28 intracellular domain, CD28 intracellular domain, 4-1BB intracellular domain and OX40 intracellular domain, and preferably it may be CD3ζ represented by the amino acid sequence of SEQ ID NO: 31.

In the present invention, it may further comprise a hinge region located between the C terminus of the BCMA-binding domain and the N terminus of the transmembrane domain, wherein the hinge region is derived from CD8α, and preferably it can be represented by the amino acid sequence of SEQ ID NO: 28. The “hinge region” generally refers to a linking region between an antigen-binding region and an immune cell Fc receptor (FcR)-binding region.

In the present invention, it may further include a signal peptide at the N-terminus of the BCMA-binding domain, and the “signal peptide” generally refers to a peptide chain for guiding protein transduction. The signal peptide may be a short peptide having a length of 5 to 30 amino acids, preferably represented by the amino acid sequence of SEQ ID NO: 27.

### Chimeric Antigen Receptor Encoding Polynucleotides and Chimeric Antigen Receptor Expression Vectors

In another aspect, the present invention relates to a polynucleotide encoding the chimeric antigen receptor (CAR).

In the present invention, the polynucleotide encoding the chimeric antigen receptor (CAR) has a polynucleotide encoding a BCMA-binding domain; a polynucleotide encoding a transmembrane domain; polynucleotides encoding the co-stimulatory domain; and a polynucleotide encoding an intracellular signal transduction domain.

A polynucleotide encoding the BCMA-binding domain may preferably be a polynucleotide encoding a 3G4 antibody or a 3G4V2 antibody, and the specific nucleotide sequence is as described above.

A polynucleotide encoding the chimeric antigen receptor (CAR) of the present invention may has a signal peptide represented by the nucleotide sequence of SEQ ID NO: 21; 3G4V2, which is an anti-BCMA antibody represented by the nucleotide sequence of SEQ ID NO: 18; a transmembrane domain represented by the nucleotide sequence of SEQ ID NO: 23; 4-1BB (costimulatory domain) represented by the nucleotide sequence of SEQ ID NO: 24; and CD3ζ (intracellular signaling domain) represented by the nucleotide sequence of SEQ ID NO: 25.

In addition, a polynucleotide encoding a hinge region may be additionally included between a polynucleotide encoding the BCMA-binding domain and a transmembrane domain, and preferably It may be a CD8 hinge region represented by the nucleotide sequence of SEQ ID NO: 22.

As used herein, the term “polynucleotide” generally refers to a nucleic acid molecule, deoxyribonucleotide or ribonucleotide, or an analog thereof, separated by any length. In some embodiments, a polynucleotide of the present invention can be prepared by (1) in-vitro amplification, such as polymerase chain reaction (PCR) amplification; (2) cloning and recombination; (3) purification such as digestion and gel electrophoretic separation; (4) synthesis such as chemical synthesis, and preferably, the isolated polynucleotide is prepared by recombinant DNA technology. In the present invention, the nucleic acid for encoding the antibody or antigen-binding fragment thereof can be prepared by various methods known in the art, including, but not limited to, restriction fragment operation of synthetic oligonucleotides or application of SOE PCR.

In another aspect, the present invention relates to a vector comprising a polynucleotide encoding the chimeric antigen receptor (CAR).

In the present invention, the term “vector (expression vector)” refers to a gene preparation including essential regulatory elements such as a promoter so that a target gene can be expressed in an appropriate host cell. A vector may be selected from one or more of a plasmid, a retroviral vector, and a lentiviral vector. Upon transformation into an appropriate host, a vector can replicate and function independently of the host genome, or in some cases can be integrated into the genome itself.

In addition, a vector may contain expression control elements that allow the coding region to be accurately expressed in a suitable host. Such regulatory elements are well known to those skilled in the art and include, for example, promoters, ribosome-binding sites, enhancers and other regulatory elements for regulating gene transcription or mRNA translation. The specific structure of the expression control sequence may vary depending on the function of the species or cell type, but generally contains 5′ non-translated sequence, and a 5′ or 3′ non-translated sequence participating in transcription initiation and translation initiation, respectively, such as TATA box, capped sequence, CAAT sequence, etc. For example, a 5′ non-transcriptional expression control sequence can include a promoter region that can include a promoter sequence for transcription and control of a functionally linked nucleic acid.

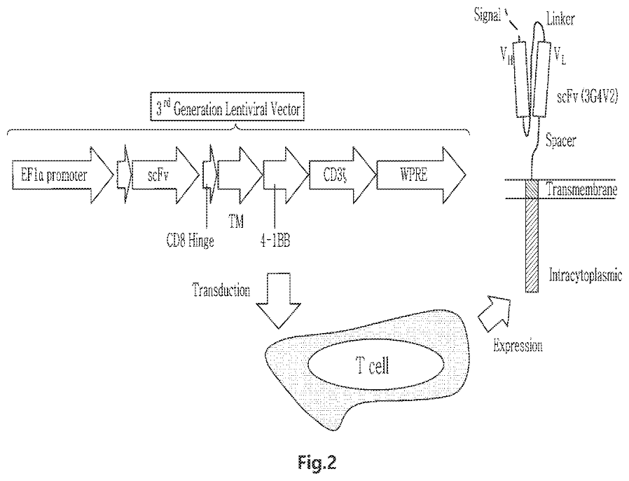

In a specific embodiment of the present invention, the vector is a recombinant virus a vector, preferably a lentivirus vector, and comprises an operably linked EF1α promoter; a polynucleotide encoding a signal peptide; a polynucleotide encoding a BCMA-binding domain; a polynucleotide encoding a transmembrane domain; and a polynucleotide encoding an intracellular signal transduction domain, and may further include a woodchuck hepatitis virus post-transcriptional regulatory element (WPRE) to increase protein expression (refer to FIG. 2).

The EF1α promoter may be represented by the nucleotide sequence of SEQ ID NO: 87, and if necessary, has 90% or more, 93% or more, 95% or more, 96% or more, 97% or more, 98% or more, or 99% or more identical sequences of the nucleotide sequence of SEQ ID NO: 19.

In addition, the promoter is operably linked to induce expression of an anti-hBCMA antibody (scFv), which is a BCMA-binding domain, wherein “operably linked” refers to a nucleic acid expression control sequence and a nucleic acid sequence encoding a target protein are functionally linked to perform a general function. The operative linkage with the recombinant vector can be prepared using genetic recombination techniques well known in the art, and site-specific DNA cleavage and ligation using enzymes generally known in the art.

Methods for introducing and expressing genes into cells are known in the art. With respect to expression vectors, vectors can be readily introduced into host cells by any method in the art. For example, an expression vector may be transferred into a host cell by physical, chemical, or biological means.

Physical methods for introducing polynucleotides into host cells include calcium phosphate precipitation, lipofection, particle bombardment, microinjection, electroporation, and the like. Methods for producing cells comprising vectors and/or exogenous nucleic acids are well known in the art. See, e.g., Sambrook et al, 2012, MOLECULAR CLONING: A LABORATORY MANUAL, volumes 1-4, Cold Spring Harbor Press, NY. A preferred method for introduction of polynucleotides into host cells is calcium phosphate transfection.

Biological methods for introducing polynucleotides into host cells include the use of DNA and RNA vectors. Viral vectors, and in particular retroviral vectors, have become the most widely used methods for inserting genes into mammalian, e.g., human cells. Other viral vectors may be derived from lentiviruses, poxviruses, herpes simplex viruses, adenoviruses and adeno-associated viruses, and the like.

Chemical means for introducing polynucleotides into host cells include colloidal dispersion systems such as macromolecular complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). State of the art in targeted delivery of nucleic acids, such as targeted other methods for delivery of polynucleotides using nanoparticles or other suitable sub-micron sized delivery systems are available.

When a non-viral delivery system is used, an exemplary delivery vehicle is a liposome. The use of lipid preparations is contemplated for the introduction of nucleic acids into host cells (in vitro, ex vivo or in vivo). In another aspect, the nucleic acid may be associated with a lipid. Nucleic acids associated with lipids may be encapsulated within the aqueous interior of the liposome, interspersed within the lipid bilayer of the liposome, attached to the liposome via a linking molecule associated with both the liposome and oligonucleotide, captured within the liposome, complexed with the liposome, dispersed in a lipid-containing solution, mixed with a lipid or combined with a lipid, contained as a suspension in a lipid, contained or complexed with micelles, or otherwise associated with a lipid. Lipid, lipid/DNA or lipid/expression a vector association composition is not limited to any particular structure in solution.

### Immune Effector Cells Expressing Chimeric Antigen Receptor (CAR)

In another aspect, the present invention relates to an immune effector cell expressing the chimeric antigen receptor (CAR), and includes a vector comprising a polynucleotide encoding a chimeric antigen receptor (CAR), or a polynucleotide encoding a chimeric antigen receptor (CAR).

In the present invention, the immune effector cell may be a mammalian-derived cell, preferably a T cell or a natural killer (NK) cell.

In the present invention, an immune effector cell expressing the chimeric antigen receptor (CAR) can be prepared by introducing the CAR vector of the present invention into an immune effector cell, for example, a T cell or NK cell.

Specifically, CAR vector can be introduced into cells by methods known in the art, such as electroporation, lipofectamine (lipofectamine 2000, Invitrogen), and the like. For example, an immune effector cell can be transformed by a lentiviral vector to integrate the viral genome carrying the CAR molecule into the host genome to ensure long-term and stable expression of the target gene. For another example, a transposon can be used to introduce a CAR transport plasmid and a transferase transport plasmid into a target cell. For another example, a CAR molecule can be added to the genome by a gene editing method (e.g., CRISPR/Cas9).

In a specific embodiment of the present invention, as shown in FIG. 2, a lentiviral vector into which a polynucleotide coating hBCMA-CAR was inserted was prepared, and the prepared vector was transformed into T cells to prepare hBCMA-CAR-T cells. The prepared hBCMA-CAR-T cells can express a chimeric antigen receptor targeting BCMA of the present invention.

An immune effector cell for the production of immune effector cell expressing a chimeric antigen receptor (CAR) can be obtained from a subject, wherein the “subject” includes a living organism (e.g., a mammal from which an immune response can be elicited). Examples of subjects include humans, dogs, cats, mice, rats, and transgenic species thereof. T cells can be obtained from numerous sources, including peripheral blood mononuclear cells, bone marrow, lymph node tissue, umbilical cord blood, thymus tissue, tissue from the site of infection, ascites, pleural effusion, splenic tissue, and tumors.

Such T cells can be obtained from blood units collected from a subject using any of a number of techniques known to those of ordinary skill in the art, for example, Ficoll™ isolation. Cells from blood are obtained by apheresis, and apheresis products typically contain T cells, monocytes, granulocytes, lymphocytes including B cells, other nucleated leukocytes, red blood cells, and platelets.

Cells collected by apheresis can be washed to remove the plasma fraction and place the cells in an appropriate buffer or medium for subsequent processing steps. T cells are isolated from peripheral blood lymphocytes by lysing red blood cells and depleting monocytes, for example by centrifugation through a PERCOLL™ gradient or by countercurrent centrifugation.

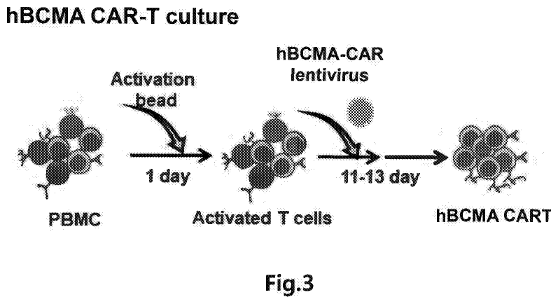

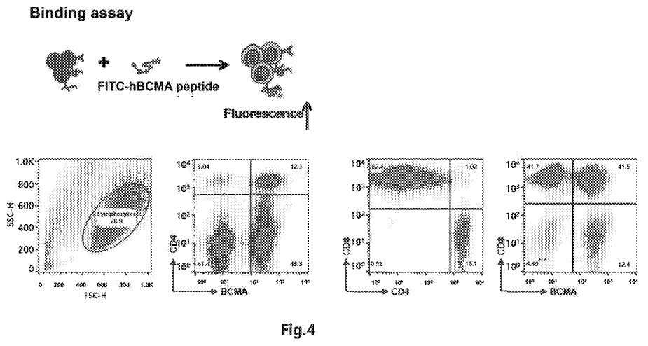

In a specific embodiment of the present invention, as shown in FIG. 3, activated T cells were isolated from peripheral blood mononuclear cells (PBMCs), and then, hBCMA-CAR lentivirus was transduced into T cells, and hBCMA-CAR-T cells were prepared. As a result of confirming the hBCMA peptide binding ability of the prepared hBCMA-CAR-T cells, as shown in FIG. 4, as the hBCMA peptide increased, it was confirmed that the hBCMA-CAR-T cells expressing CD4 or CD8 binding to hBCMA increased. This means that the hBCMA-CAR-T cells prepared in the present invention effectively can bind BCMA.

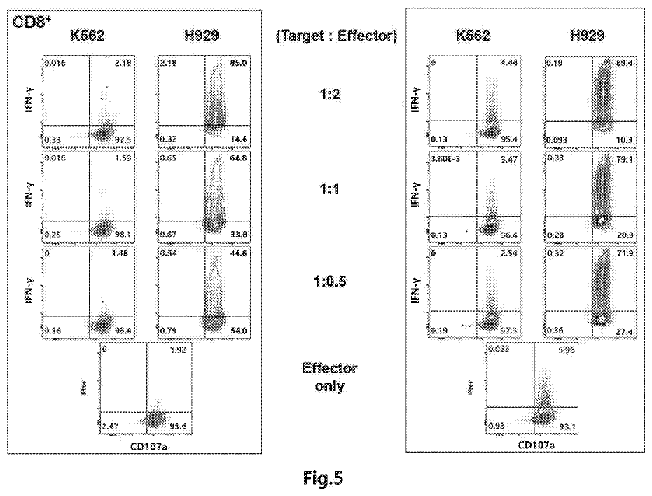

In a specific embodiment of the present invention, in order to confirm the activation of hBCMA-CAR-T cells, the expression level of IFNγ and CD107a by 3G4V2-CAR-T cells in the presence of target cells was checked. As a result, as shown in FIG. 5, T cells were not activated in BCMA-expressing K562 cells, whereas T cells were activated in the presence of BCMA-expressing H929 cells to increase IFNγ expression.

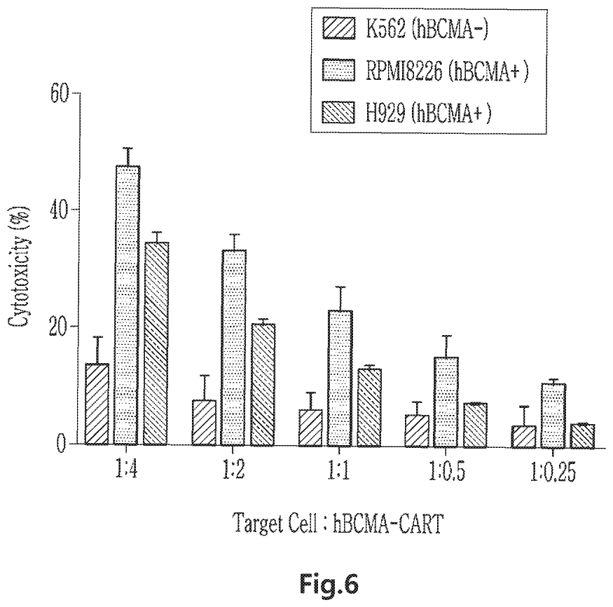

In a specific embodiment of the present invention, as a result of confirming the killing effect of the target cells by the hBCMA-CAR-T cells, as shown in FIG. 6, it was confirmed that hBCMA-CAR-T cells specifically killed RPM18226 cells and H929 cells expressing BCMA.

That is, the chimeric antigen receptor and CAR-T cell targeting BCMA of the present invention can be usefully used as a composition for preventing or treating diseases related to B cell expression or BCMA expression.

### Composition for Preventing or Treating Diseases Related to BCMA Expression

In another aspect, the present invention relates to a pharmaceutical composition for preventing or treating diseases related to B cells, including immune effector cells expressing a chimeric antigen receptor targeting BCMA.

In another aspect, the present invention relates to a pharmaceutical composition for preventing or treating diseases related to B cells comprising an antibody targeting BCMA.

A disease associated with B cells or a disease associated with BCMA (wild-type or mutant BCMA) expression may be cancer, malignancy or an autoimmune disease. Preferably multiple myeloma, hematological cancer, non-Hodgkin It may be non-Hodgkin's lymphoma, autoantibody-dependent autoimmune disease, systemic lupus erythematosus (SLE) or rheumatoid arthritis.

The pharmaceutical composition may further include a pharmaceutically acceptable carrier. For oral administration, binders, lubricants, disintegrants, excipients, solubilizers, dispersants, stabilizers, suspending agents, pigments, fragrances, etc. can be used. In the case of an injection, a buffer, a preservative, an analgesic, a solubilizer, an isotonic agent, a stabilizer, and the like may be mixed and used. In the case of topical administration, bases, excipients, lubricants, preservatives, etc. can be used.

The formulation of the pharmaceutical composition may be prepared in various ways by mixing with the above-described pharmaceutically acceptable carrier. For example, in the case of oral administration, it may be prepared in the form of tablets, troches, capsules, elixirs, suspensions, syrups, wafers, and the like, and in the case of injections, it may be prepared in the form of unit dose ampoules or multiple doses.

In addition, the pharmaceutical composition may include a surfactant capable of improving membrane permeability. Such surfactants may be steroid-derived or cationic lipids such as N-[1-(2,3-dioleoyl) propyl-N,N,N-trimethylammonium chloride (DOTMA), or cholesterol hemisuccinate, various compounds such as phosphatidyl glycerol, but is not limited thereto.

In addition, the present invention provides a method for preventing or treating cancer, comprising administering the pharmaceutical composition according to the present invention to a subject. The pharmaceutical composition comprising the hBCMA-CAR-T cell or anti-hBCMA antibody may be administered in a pharmaceutically effective amount to prevent or treat a disease associated with B cells. It may vary depending on various factors such as the type of disease, the age and weight of the patient, the characteristics and severity of symptoms, the type of current treatment, the number of treatments, the dosage form, and route, and may be easily determined by experts in the field.

The pharmaceutical composition may be administered together or sequentially with the aforementioned pharmacological or physiological ingredient, and may be administered in combination with an additional conventional therapeutic agent, or may be administered sequentially or simultaneously with the conventional therapeutic agent. Such administration may be single or multiple administrations. Taking all of the above factors into consideration, it is important to administer an amount that can obtain the maximum effect with a minimum amount without side effects, which can be easily determined by those skilled in the art.

As used herein, the term “subject” refers to a mammal suffering from or at risk of a condition or disease that can be alleviated, suppressed or treated by administering the pharmaceutical composition, and preferably refers to a human.

As used herein, the term ‘administration’ refers to providing the pharmaceutical composition of the present invention to an individual by any suitable method. The pharmaceutical composition of the present invention provides an amount of an active ingredient or pharmaceutical composition that induces a biological or medical response in a tissue system, animal or human as thought by a researcher, veterinarian, doctor or other clinician, that is, alleviation of symptoms of a disease or disorder to be treated. It can be administered in a therapeutically effective amount, which is an amount that induces It is apparent to those skilled in the art that the therapeutically effective dosage and frequency of administration for the pharmaceutical composition of the present invention will vary depending on the desired effect. Therefore, the optimal dosage to be administered can be easily determined by those skilled in the art, and the type of disease, the severity of the disease, the content of active ingredients and other components contained in the composition, the type of formulation, the age, weight, and general health of the patient, gender and diet, administration time, administration route and secretion rate of the composition, treatment period, and various factors including concurrently used drugs. The pharmaceutical composition of the present invention may be administered in an amount of 1 to 10,000 mg/kg/day, may be administered once a day, or may be administered in divided doses.

Hereinafter, preferred examples are presented to help the understanding of the present invention. However, the following examples are only provided for easier understanding of the present invention, and the contents of the present invention are not limited by the following examples.

### Example 1: Preparation and Selection of the Antibody Targeting BCMA

In order to select the BCMA peptide-specific antibody, a hybridoma producing an antibody binding to BCMA was prepared and the antibody was selected.

First, mice were immunized using a BCMA peptide (SEQ ID NO: 19; Acrobiosystems Inc. cat #BCA-H522y) by a method known in the art, and then splenocytes were extracted and hybridomas cells were prepared through cell fusion with mouse myeloma cells.

Mouse myeloma cells used for cell fusion cannot survive in HAT medium because they do not have HGPRT (HypoxanthineGuanidine-Phosphoribosyl-Transferase), but hybridomas can survive in HAT medium by fusion with splenocytes. Since only hybridomas could be grown using this, it is usually grown in HAT medium until hybridomas were established.

The limiting dilution method was used to select hybridomas that produced the antibody that binds to BCMA. First, it was made to be less than one cell per 96 well, and then clones proliferated from one cell were selected. The above process was repeated three times to select a hybridoma producing an antibody binding to BCMA.

The selected hybridoma was named 3G4 antibody (anti-hBCMA monoclonal antibody), and the amino acid sequence was analyzed. The sequence information for the heavy chain variable region and the light chain variable region of the antibody according to the sequencing results is shown in Table 1, and the underlined portions in Table 1 indicate the complementarity determining region (CDR).

TABLE 1
Sequence information of 3G4 antibody

| 3G4 | sequence information | SEQ ID NO: |
|---|---|---|
| Heavy chain | GYTFSYV | SEQ ID NO: 1 |
| variable region | | |
| CDR1 | | |
| Heavy chain | IIPYNDGT | SEQ ID NO: 2 |
| variable region | | |
| CDR2 | | |
| Heavy chain | ARWNWDGYFDV | SEQ ID NO: 3 |
| variable region | | |
| CDR3 | | |
| Light chain | KSLLHSNGITY | SEQ ID NO: 4 |
| variable region CDR1 | | |
| Light chain | QMS | SEQ ID NO: 5 |
| variable region CDR2 | | |
| Light chain | TQNLELPFT | SEQ ID NO: 6 |
| variable region CDR3 | | |
| Amino acid | QVQLKESGPELVKPGASVKMSCKAS GYTFTSYV | SEQ ID NO: 7 |
| sequence of heavy | MHWVKQKPGQGLEWIGY IIPYNDGT | |
| chain variable | KYNEKFKGKATLTSDKSSSTAYMELSSLTSEDSALYYC | |
| region | ARWNWDGYFDV WGAGTTVTVSS | |
| Amino acid | DIVMTQAAFSNPVTLGTSASISCRSS KSLLHSNGITY | SEQ ID NO: 8 |
| sequence of light | LYWYLQKPGQSPQLLIY QMS | |
| chain variable | NLASGVPDRFSSSGSGTDFTLRISRVEAEDVGVFYC | |
| region | TQNLELPFTF GSGTKLEIK | |
| Nucleotide | CAGGTGCAGCTGAAGGAGTCTGGACCTGAGCTGG | SEQ ID NO: 9 |
| sequence of heavy | TAAAGCCTGGGGCTTCAGTGAAGATGTCCTGCAAG | |
| chain variable region | GCTTCTGGATACACATTCACTAGCTATGTTATGCAC | |
| | TGGGTGAAGCAGAAGCCTGGGCAGGGCCTTGAGT | |
| | GGATTGGATATATTATTCCTTACAATGATGGTACTA | |
| | AGTACAATGAGAAGTTCAAAGGCAAGGCCACACTG | |
| | ACTTCAGACAAATCCTCCAGTACAGCCTACATGGAG | |
| | CTCAGCAGCCTGACCTCTGAGGACTCTGCGCTCTAT | |
| | TACTGTGCAAGATGGAACTGGGACGGGTACTTCGA | |
| | TGTCTGGGGCGCAGGGACCACGGTCACCGTCTCCT | |
| | CA | |
| Nucleotide | GATATTGTGATGACGCAGGCTGCATTCTCCAATCCA | SEQ ID NO: 10 |
| sequence of light | GTCACTCTTGGAACATCAGCTTCCATCTCCTGCAGG | |
| chain variable | TCTAGTAAGAGTCTCCTACATAGTAATGGCATCACT | |
| region | TATTTGTATTGGTATCTGCAGAAGCCAGGCCAGTCT | |
| | CCTCAGCTCCTGATTTATCAGATGTCCAACCTTGCCT | |
| | CAGGAGTCCCAGACAGGTTCAGTAGCAGTGGGTCA | |
| | GGAACTGATTTCACACTGAGAATCAGCAGAGTGGA | |
| | GGCTGAGGATGTGGGTGTTTTTTACTGTACTCAAA | |
| | ATCTAGAACTTCCATTCACGTTCGGCTCGGGGACAA | |
| | AGTTGGAAATAAAA | |

As shown in Table 1, it was confirmed that 3G4 antibody had a heavy chain variable region comprising a CDR1 region represented by the amino acid sequence of SEQ ID NO: 1 (GYTFTSYV), a CDR2 region represented by the amino acid sequence of SEQ ID NO: 2 (IIPYNDGT), and a CDR3 region represented by the amino acid sequence of SEQ ID NO: 3 (ARWNWDGYFDV); and a light chain variable comprising a CDR1 region represented by the amino acid sequence of SEQ ID NO: 4 (KSLLHSNGITY), a CDR2 region represented by the amino acid sequence of SEQ ID NO: 5 (QMS), and a CDR3 region represented by the amino acid sequence of SEQ ID NO: 6 (TONLELPFT).

Specifically, it was confirmed that 3G4 antibody had a heavy chain variable region represented by the amino acid sequence of SEQ ID NO: 7 and a light chain variable region represented by the amino acid sequence of SEQ ID NO: 8; and the heavy chain variable region of 3G4 antibody was encoded by the nucleotide sequence of SEQ ID NO: 9 and the light chain variable region of the 3G4 antibody was encoded by the nucleotide sequence of SEQ ID NO: 10.

### Example 2: Preparation of Humanized Antibody Based on 3G4 Antibody

A humanized antibody was prepared in which the 3G4 antibody selected in Example 1 was changed to a structure corresponding to a human.

Specifically, a humanized antibody was prepared from the mouse 3G4 antibody using a CDR-grafting method that replaces the CDRs of a human antibody with the CDRs of a mouse antibody that binds to BCMA with the germline sequence of a human antibody as a frame. The humanized antibody was named 3G4V2 and the amino acid sequence was analyzed. The sequence information on the heavy chain variable region and the light chain variable region of the antibody according to the sequencing results is shown in Table 2, and the underlined parts in Table 2 indicate the complementarity determining region (CDR).

TABLE 2
Sequence information of 3G4V2 antibody

| 3G4V2 | sequence information | SEQ ID NO: |
|---|---|---|
| Heavy chain | GYTFSYV | SEQ ID NO: 1 |
| variable region | | |
| CDR1 | | |
| Heavy chain | IIPYNDGT | SEQ ID NO: 2 |
| variable region | | |
| CDR2 | | |
| Heavy chain | ARWNWDGYFDV | SEQ ID NO: 3 |
| variable region | | |
| CDR3 | | |
| Light chain | KSLLHSNGITY | SEQ ID NO: 4 |
| variable region CDR1 | | |
| Light chain | QMS | SEQ ID NO: 5 |
| variable region CDR2 | | |
| Light chain | TQNLELPFT | SEQ ID NO: 6 |
| variable region CDR3 | | |
| Amino acid | QVQLVQSGAEVKKPGASVKVSCKAS GYTFTSYV | SEQ ID NO: 13 |
| sequence of heavy | MHWVRQAPGQRLEWIGY IIPYNDGT | |
| chain variable | KYNEKFQGRVTLTSDKSSSTAYMELSSLRSEDTAV YYC | |
| region | ARWNWDGYFDV WGQGTTVTVSS | |
| Amino acid | DIVMTQSPLSLPVTPGEPASISCRSS KSLLHSNGITY | SEQ ID NO: 14 |
| sequence of light | LYWYLQKPGQSPQLLIY QMS | |
| chain variable | NRASGVPDRFSSSGSGTDFTLKISRVEAEDVGVYYC | |
| region | TQNLELPFT FGQGTKLEIK | |
| Nucleotide | CAGGTGCAGCTGGTGCAGAGCGGCGCCGAGGTGA | SEQ ID NO: 15 |
| sequence of heavy | AGAAGCCGGGTGCTTCCGTGAAGGTGTCCTGTAAG | |
| chain variable | GCCTCTGGCTACACCTTCACCAGCTACGTGATGCAT | |
| region | TGGGTCCGCCAGGCCCCCGGACAGCGCCTGGAGT | |
| | GGATCGGTTACATCATCCCGTACAACGACGGCACT | |
| | AAGTACAACGAGAAATTTCAGGGCCGAGTGACCCT | |
| | GACCTCCGACAAATCCAGCTCGACCGCCTACATGG | |
| | AGCTGTCTTCTCTGCGCTCGGAGGACACCGCGGTTT | |
| | ATTACTGTGCTCGTTGGAACTGGGATGGCTATTTCG | |
| | ACGTGTGGGGCCAGGGAACGACCGTCACCGTGTC | |
| | GTCC | |
| Nucleotide | GACATCGTGATGACCCAGAGCCCTTTGTCTCTTCCT | SEQ ID NO: 16 |
| sequence of light | GTCACTCCGGGGGAGCCAGCTTCTATCTCATGCCG | |
| chain variable | ATCTTCCAAGAGCCTGCTGCACTCAAATGGCATCAC | |
| region | CTACCTCTATTGGTACCTGCAGAAGCCCGGGCAATC | |
| | CCCTCAGTTGCTCATCTATCAGATGTCTAACCGCGC | |
| | CTCCGGTGTCCCCGACCGCTTCAGCTCCTCTGGCTC | |
| | CGGCACCGACTTTACTCTGAAGATATCCCGCGTGG | |
| | AGGCCGAAGATGTGGGCGTGTACTACTGCACTCAG | |
| | AACCTGGAACTGCCCTTCACCTTCGGCCAGGGCAC | |
| | CAAGCTGGAGATCAAG | |

As shown in Table 2, it was confirmed that 3G4V2 antibody had a heavy chain variable region represented by the amino acid sequence of SEQ ID NO: 13 and a light chain variable region represented by the amino acid sequence of SEQ ID NO: 14; and the heavy chain variable region of 3G4V2 antibody was encoded by the nucleotide sequence of SEQ ID NO: 15 and the light chain variable region of 3G4V2 antibody was encoded by the nucleotide sequence of SEQ ID NO: 16.

### Example 3: Confirmation of BCMA Binding Ability of Anti-BCMA Antibody

In the present invention, it was confirmed whether the humanized anti-BCMA antibody, 3G4V2, prepared in Example 2 can specifically target BCMA.

First, to produce 3G4V2 antibody, the heavy chain variable region represented by the nucleotide sequence of SEQ ID NO: 15 was cloned into a heavy chain antibody expression vector of human antibody IgG1, and the light chain variable region represented by the nucleotide sequence of SEQ ID NO: 16 was cloned into was cloned into a light chain antibody expression vector of human antibody kappa; and each expression vector was co-transfected with CHO cells to produce an antibody. The produced antibody was purified using a Protein A column (Thermo Fisher, cat #20356).

Then, 1 mg each of the purified antibody was treated with A549 cells and hBCMA-expressing A549 cells, followed by treatment with a secondary antibody (Biolegend, cat #409304) specific for human IgG Fc. After reacting with the antibody, PE fluorescence of the secondary antibody was measured by flow cytometry.

As a result, as shown in FIG. 1, it was confirmed that binding to 3G4V2 was not confirmed in A549 cells not expressing hBCMA, but the binding capacity of A549 cells expressing hBCMA and 3G4V2 was increased to 60.1%.

### Example 4: Construction of a Chimeric Antigen Receptor (CAR) Expression Vector Targeting BCMA

In the present invention, a lentiviral vector (hBCMA-CAR lentivirus) expressing a chimeric antigen receptor (CAR) targeting BCMA containing 3G4V2 (humanized anti-BCMA antibody), prepared in Example 2, was prepared.

As shown in the schematic diagram of FIG. 3, CAR DNA composed of: EF1α promoter (SEQ ID NO: 20); a polynucleotide encoding a signal peptide (SEQ ID NO: 21); a polynucleotide encoding a BCMA-binding domain (SEQ ID NO: 18); a polynucleotide encoding a CD8 hinge region (SEQ ID NO: 22); a polynucleotide encoding a transmembrane domain (SEQ ID NO:23); a polynucleotide encoding 4-1BB (costimulatory domain) (SEQ ID NO: 24); a polynucleotide encoding CD3ζ intracellular signaling domain (SEQ ID NO: 25); and a polynucleotide encoding WPRE (SEQ ID NO: 26); was synthesized in vitro and inserted into a third-generation lentiviral vector.

Lenti-X 293T cells (co-After transfection) were co-infected with lentiviral vector and three vectors of pMDLg/pRRE (Addgene, cat ##12251), pMD2.G (Addgene, cat ##12259) & pRSV-Rev (Addgene, cat ##12253), and then hBCMA-CAR lentivirus was produced. For co-transfection, three vectors and Lenti-X 293T cells were incubated for 6 hours using Lipofectamine 3000 transfection kit (Invitrogen, cat #L3000-015) and Opti-MEM+GlutaMAX (gibco, cat #51985-034) media.

### Example 5: Preparation of hBCMA-CAR-T Cell

In the present invention, the hBCMA-CAR lentiviral vector prepared in Example 4 was transformed into T cells, hBCMA-CAR-T cells (or 3G4V2-CAR-T cells) were prepared.

Specifically, as shown in FIG. 3, after separating peripheral blood mononuclear cells (PBMC) from blood, T cell activation beads (T cell activation beads; Miltenyl Biotec, cat #130-091-441) was used to activate T cells. hBCMA-CAR-T cells were prepared by transducing the activated T cells with the hBCMA-CAR lentivirus prepared in Example 4, and Lenti-boost-p was used to increase the transduction efficiency.

hBCMA peptide binding capacity of hBCMA-CAR-T cells was confirmed by flow cytometry. The hBCMA-CAR-T cells prepared above were reacted with FITC-hBCMA protein with anti-CD3, anti-CD4, and anti-CD8 antibodies, and then fluorescence intensity was measured using a FACS machine. In the course of the analysis, CD3 expressing cells were used as T cells, and the level of FITC expression in T cells was confirmed.

As a result, as shown in FIG. 4, it was confirmed that hBCMA-CAR-T cells expressing CD4 or CD8 binding to hBCMA increased as the hBCMA peptide increased. This means that the hBCMA-CAR-T cells prepared in the present invention effectively bind to BCMA.

### Example 6: Confirmation of hBCMA-CAR-T Cell Activation by hBCMA Peptide

In the present invention, in order to check whether the hBCMA-CAR-T cells prepared in Example 5 are activated by the BCMA peptide, IFNγ and CD107a expression levels by hBCMA-CAR-T cells in the presence of target cells were confirmed.

As target cells, K562 cells that do not express BCMA (ATCC, cat #CCL-243) and H929 cells that express BCMA (ATCC, cat #CRL-9068) were used. After reacting hBCMA-CAR-T cells and target cells at a ratio of 2:1, 1:1. 0.5:1, 0:1 for a certain period of time, FACS measurement was performed by staining with surface & intra antibody (BCMA protein, INF-r, CD107a, CD3, CD4, CD8 staining). The levels of IFNγ and CD107a expressions of hBCMA-CAR-T reacted with target cells based on 0:1 (CAR-T only) were confirmed.

As a result, as shown in FIG. 5, it was confirmed that T cells were not activated in K562 cells not expressing BCMA, whereas T cells were activated in the presence of BCMA-expressing H929 cells to increase IFNγ expression.

### Example 7: Confirmation of the Killing Effect of hBCMA-CAR-T Cells on BCMA-Expressing Cells

In the present invention, the killing effect of target cells by hBCMA-CAR-T cells was confirmed.

As target cells, K562 cells that do not express BCMA, and RPMI8226 cells and H929 cells that express BCMA were used. After reacting hBCMA-CAR-T cells and target cells at a ratio of 1:4, 1:2, 1:1, 1:0.5 and 1:0.25, luminescence (CytoTox-Glo Cytotoxicity Assay, Promega, cat #G9291) was measured. From the measured value, the degree of cell death was calculated using Equation 1 below.

% Cytotoxicity=[(Experimental−Effector Spontaneous−Target Spontaneous)/(Target Maximum-Target Spontaneous)]×100 [Equation 1]
Experimental: Luminescence value derived from the medium of the target cell and CAR-T cell complex culture
Effector Spontaneous: Luminescence value derived from the medium of CAR-T cells only
Target Spontaneous: Luminescence value derived from the medium of target cells only
Target Maximum: Luminescence value derived from 100% lysis of target cells (using Lysis Reagent)

As a result, as shown in FIG. 6, it was confirmed that hBCMA-CAR-T cells showed a specific killing effect on RPMI8226 cells and H929 cells expressing BCMA.

That is, chimeric antigen receptor and CAR-T cell targeting BCMA of the present invention can be usefully used as a composition for preventing or treating diseases related to B cell expression or BCMA expression.

### INDUSTRIAL APPLICABILITY

It was confirmed that the antibody selected in the present invention specifically recognized BCMA-expressing cells, and the chimeric antigen receptor (CAR) and CAR-T cells targeting BCMA using the established antibody effectively bound BCMA. In addition, it was confirmed that the activation of CAR-T cells bound to BCMA was made.

In addition, since it was confirmed that the CAR-T cells of the present invention effectively killed BCMA-expressing cells, BCMA-specific antibody, BCMA-targeting chimeric antigen receptor, and CAR-T cells of the present invention can be usefully used for preventing or treating diseases related to B cell expression or BCMA expression.

1. An antibody specifically binding to BCMA (B-cell maturation antigen) or fragment thereof, comprising:
a heavy chain variable region including a CDR1 region represented by the amino acid sequence of SEQ ID NO: 1, a CDR2 region represented by the amino acid sequence of SEQ ID NO: 2, and a CDR3 region represented by the amino acid sequence of SEQ ID NO: 3; and
a light chain variable region including a CDR1 region represented by the amino acid sequence of SEQ ID NO: 4, a CDR2 region represented by the amino acid sequence of SEQ ID NO: 5, and a CDR3 region represented by the amino acid sequence of SEQ ID NO: 6.

2. The antibody specifically binding to BCMA or fragment thereof according to claim 1, wherein the antibody comprises a heavy chain variable region represented by the amino acid sequence of SEQ ID NO: 7 and a light chain variable region represented by the amino acid sequence of SEQ ID NO: 8; or a heavy chain variable region represented by the amino acid sequence of SEQ ID NO: 13 and a light chain variable region represented by the amino acid sequence of SEQ ID NO: 14.

3. A chimeric antigen receptor (CAR) comprising: a BCMA-binding domain; a transmembrane domain; a costimulatory domain; and an intracellular signal transduction domain,
wherein the BCMA-binding domain includes an antibody specifically binding to BCMA or fragment thereof comprising:
a heavy chain variable region comprising a CDR1 region represented by the amino acid sequence of SEQ ID NO: 1, a CDR2 region represented by the amino acid sequence of SEQ ID NO: 2 and a CDR3 region represented by the amino acid sequence of SEQ ID NO: 3; and
a light chain variable region comprising a CDR1 region represented by the amino acid sequence of SEQ ID NO: 4, a CDR2 region represented by the amino acid sequence of SEQ ID NO: 5, and a CDR3 region represented by the amino acid sequence of SEQ ID NO: 6.

4. The chimeric antigen receptor according to claim 3, wherein the transmembrane domain is a protein selected from the group consisting of CD8a, CD4, CD28, CD137, CD80, CD86, CD152 and PD1.

5. The chimeric antigen receptor according to claim 3, wherein the costimulatory domain is a protein selected from the group consisting of CD28, 4-1BB, OX-40 and ICOS.

6. The chimeric antigen receptor of claim 3, wherein the signaling domain is CD3ζ.

7. The chimeric antigen receptor according to claim 3, further comprising a hinge region between the C terminus of the BCMA-binding domain and the N terminus of the transmembrane domain.

8. A polynucleotide encoding the chimeric antigen receptor (CAR) of any one of claims 3 to 7.

9. A vector comprising a polynucleotide encoding the chimeric antigen receptor (CAR) of any one of claims 3 to 7.

10. The vector according to claim 9, wherein the vector is a plasmid, a retroviral vector, or a lentiviral.

11. An immune effector cell comprising a polynucleotide encoding the chimeric antigen receptor (CAR) of any one of claims 3 to 7 or a vector comprising the polynucleotide.

12. The immune effector cell according to claim 11, wherein the immune effector cell is a T cell or a Natural Killer cell.

13. A pharmaceutical composition for preventing or treating a disease relating to BCMA expression comprising the immune effector cell of claim 11.

14. The pharmaceutical composition for preventing or treating a disease relating to BCMA expression according to claim 13, wherein the disease relating to BCMA expression is multiple myeloma, hematologic cancer, non-Hodgkin's lymphoma, autoantibody-dependent autoimmune disease, systemic lupus erythematosus (SLE) or rheumatoid arthritis.


SEQUENCE LISTING (32 sequences)

SEQ ID NO: 1 - protein, 8 aa, synthetic construct
  REGION 1..8: note = 3G4_VH_CDR1
GYTFTSYV

SEQ ID NO: 2 - protein, 8 aa, synthetic construct
  REGION 1..8: note = 3G4_VH_CDR2
IIPYNDGT

SEQ ID NO: 3 - protein, 11 aa, synthetic construct
  REGION 1..11: note = 3G4_VH_CDR3
ARWNWDGYFDV

SEQ ID NO: 4 - protein, 11 aa, synthetic construct
  REGION 1..11: note = 3G4_VL_CDR1
KSLLHSNGITY

SEQ ID NO: 5 - skipped

SEQ ID NO: 6 - protein, 9 aa, synthetic construct
  REGION 1..9: note = 3G4_VL_CDR3
TQNLELPFT

SEQ ID NO: 7 - protein, 118 aa, synthetic construct
  REGION 1..118: note = 3G4_VH chain
QVQLKESGPELVKPGASVKMSCKASGYTFTSYVMHWVKQKPGQGLEWIGYIIPYNDGTKYNEKFKGKATLTSDKSSSTAYMELSSLTSEDSALYYCARWNWDGYFDVWGAGTTVTVSS

SEQ ID NO: 8 - protein, 112 aa, synthetic construct
  REGION 1..112: note = 3G4_VL chain
DIVMTQAAFSNPVTLGTSASISCRSSKSLLHSNGITYLYWYLQKPGQSPQLLIYQMSNLASGVPDRFSSSGSGTDFTLRISRVEAEDVGVFYCTQNLELPFTFGSGTKLEIK

SEQ ID NO: 9 - DNA, 354 bp, synthetic construct
  misc_feature 1..354: note = 3G4_VH chain
  source 1..354: mol_type = other DNA
caggtgcagctgaaggagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatggtactaagtacaatgagaagttcaaaggcaaggccacactgacttcagacaaatcctccagtacagcctacatggagctcagcagcctgacctctgaggactctgcgctctattactgtgcaagatggaactgggacgggtacttcgatgtctggggcgcagggaccacggtcaccgtctcctca

SEQ ID NO: 10 - DNA, 336 bp, synthetic construct
  misc_feature 1..336: note = 3G4_VL chain
  source 1..336: mol_type = other DNA
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgttttttactgtactcaaaatctagaacttccattcacgttcggctcggggacaaagttggaaataaaa

SEQ ID NO: 11 - protein, 245 aa, synthetic construct
  REGION 1..245: note = 3G4_scFv
DIVMTQAAFSNPVTLGTSASISCRSSKSLLHSNGITYLYWYLQKPGQSPQLLIYQMSNLASGVPDRFSSSGSGTDFTLRISRVEAEDVGVFYCTQNLELPFTFGSGTKLEIKGGGGSGGGGSGGGGSQVQLKESGPELVKPGASVKMSCKASGYTFTSYVMHWVKQKPGQGLEWIGYIIPYNDGTKYNEKFKGKATLTSDKSSSTAYMELSSLTSEDSALYYCARWNWDGYFDVWGAGTTVTVSS

SEQ ID NO: 12 - DNA, 735 bp, synthetic construct
  misc_feature 1..735: note = 3G4_scFv
  source 1..735: mol_type = other DNA
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgttttttactgtactcaaaatctagaacttccattcacgttcggctcggggacaaagttggaaataaaaggtggtggtggttcgggtggtggtggttcgggtggtggtggttcgcaggtgcagctgaaggagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatggtactaagtacaatgagaagttcaaaggcaaggccacactgacttcagacaaatcctccagtacagcctacatggagctcagcagcctgacctctgaggactctgcgctctattactgtgcaagatggaactgggacgggtacttcgatgtctggggcgcagggaccacggtcaccgtctcctca

SEQ ID NO: 13 - protein, 118 aa, synthetic construct
  REGION 1..118: note = 3G4V2_VH chain
QVQLVQSGAEVKKPGASVKVSCKASGYTFTSYVMHWVRQAPGQRLEWIGYIIPYNDGTKYNEKFQGRVTLTSDKSSSTAYMELSSLRSEDTAVYYCARWNWDGYFDVWGQGTTVTVSS

SEQ ID NO: 14 - protein, 112 aa, synthetic construct
  REGION 1..112: note = 3G4V2_VL chain
DIVMTQSPLSLPVTPGEPASISCRSSKSLLHSNGITYLYWYLQKPGQSPQLLIYQMSNRASGVPDRFSSSGSGTDFTLKISRVEAEDVGVYYCTQNLELPFTFGQGTKLEIK

SEQ ID NO: 15 - DNA, 354 bp, synthetic construct
  misc_feature 1..354: note = 3G4V2_VH chain
  source 1..354: mol_type = other DNA
caggtgcagctggtgcagagcggcgccgaggtgaagaagccgggtgcttccgtgaaggtgtcctgtaaggcctctggctacaccttcaccagctacgtgatgcattgggtccgccaggcccccggacagcgcctggagtggatcggttacatcatcccgtacaacgacggcactaagtacaacgagaaatttcagggccgagtgaccctgacctccgacaaatccagctcgaccgcctacatggagctgtcttctctgcgctcggaggacaccgcggtttattactgtgctcgttggaactgggatggctatttcgacgtgtggggccagggaacgaccgtcaccgtgtcgtcc

SEQ ID NO: 16 - DNA, 336 bp, synthetic construct
  misc_feature 1..336: note = 3G4V2_VL chain
  source 1..336: mol_type = other DNA
gacatcgtgatgacccagagccctttgtctcttcctgtcactccgggggagccagcttctatctcatgccgatcttccaagagcctgctgcactcaaatggcatcacctacctctattggtacctgcagaagcccgggcaatcccctcagttgctcatctatcagatgtctaaccgcgcctccggtgtccccgaccgcttcagctcctctggctccggcaccgactttactctgaagatatcccgcgtggaggccgaagatgtgggcgtgtactactgcactcagaacctggaactgcccttcaccttcggccagggcaccaagctggagatcaag

SEQ ID NO: 17 - protein, 245 aa, synthetic construct
  REGION 1..245: note = 3G4V2_scFv
DIVMTQSPLSLPVTPGEPASISCRSSKSLLHSNGITYLYWYLQKPGQSPQLLIYQMSNRASGVPDRFSSSGSGTDFTLKISRVEAEDVGVYYCTQNLELPFTFGQGTKLEIKGGGGSGGGGSGGGGSQVQLVQSGAEVKKPGASVKVSCKASGYTFTSYVMHWVRQAPGQRLEWIGYIIPYNDGTKYNEKFQGRVTLTSDKSSSTAYMELSSLRSEDTAVYYCARWNWDGYFDVWGQGTTVTVSS

SEQ ID NO: 18 - DNA, 735 bp, synthetic construct
  misc_feature 1..735: note = 3G4V2_scFv
  source 1..735: mol_type = other DNA
gacatcgtgatgacccagagccctttgtctcttcctgtcactccgggggagccagcttctatctcatgccgatcttccaagagcctgctgcactcaaatggcatcacctacctctattggtacctgcagaagcccgggcaatcccctcagttgctcatctatcagatgtctaaccgcgcctccggtgtccccgaccgcttcagctcctctggctccggcaccgactttactctgaagatatcccgcgtggaggccgaagatgtgggcgtgtactactgcactcagaacctggaactgcccttcaccttcggccagggcaccaagctggagatcaagggcggtggtggctccggcggagggggttctggaggcggcggctcccaggtgcagctggtgcagagcggcgccgaggtgaagaagccgggtgcttccgtgaaggtgtcctgtaaggcctctggctacaccttcaccagctacgtgatgcattgggtccgccaggcccccggacagcgcctggagtggatcggttacatcatcccgtacaacgacggcactaagtacaacgagaaatttcagggccgagtgaccctgacctccgacaaatccagctcgaccgcctacatggagctgtcttctctgcgctcggaggacaccgcggtttattactgtgctcgttggaactgggatggctatttcgacgtgtggggccagggaacgaccgtcaccgtgtcgtcc

SEQ ID NO: 19 - protein, 54 aa, synthetic construct
  REGION 1..54: note = BCMA peptide
MLQMAGQCSQNEYFDSLLHACIPCQLRCSSNTPPLTCQRYCNASVTNSVKGTNA

SEQ ID NO: 20 - DNA, 1178 bp, synthetic construct
  misc_feature 1..1178: note = EF1 promoter
  source 1..1178: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 63 bp, synthetic construct
  misc_feature 1..63: note = signal peptide
  source 1..63: mol_type = other DNA
atggccttaccagtgaccgccttgctcctgccgctggccttgctgctccacgccgccaggccg

SEQ ID NO: 22 - DNA, 135 bp, synthetic construct
  misc_feature 1..135: note = CD8 Hinge
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 23 - DNA, 72 bp, synthetic construct
  misc_feature 1..72: note = transmembrane domain
  source 1..72: mol_type = other DNA
atctacatctgggcgcccttggccgggacttgtggggtccttctcctgtcactggttatcaccctttactgc

SEQ ID NO: 24 - DNA, 126 bp, synthetic construct
  misc_feature 1..126: note = 4-1BB
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 25 - DNA, 336 bp, synthetic construct
  misc_feature 1..336: note = CD3_zeta
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - DNA, 591 bp, synthetic construct
  misc_feature 1..591: note = WPRE
  source 1..591: mol_type = other DNA
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 27 - protein, 21 aa, synthetic construct
  REGION 1..21: note = signal peptide
MALPVTALLLPLALLLHAARP

SEQ ID NO: 28 - protein, 45 aa, synthetic construct
  REGION 1..45: note = CD8 Hinge
TTTPAPRPPTPAPTIASQPLSLRPEACRPAAGGAVHTRGLDFACD

SEQ ID NO: 29 - protein, 24 aa, synthetic construct
  REGION 1..24: note = transmembrane domain
IYIWAPLAGTCGVLLLSLVITLYC

SEQ ID NO: 30 - protein, 42 aa, synthetic construct
  REGION 1..42: note = 4-1BB
KRGRKKLLYIFKQPFMRPVQTTQEEDGCSCRFPEEEEGGCEL

SEQ ID NO: 31 - protein, 112 aa, synthetic construct
  REGION 1..112: note = CD3_zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 2594 bp, synthetic construct
  misc_feature 1..2594: note = CAR for lentivirus insertion
  source 1..2594: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatccgacatcgtgatgacccagagccctttgtctcttcctgtcactccgggggagccagcttctatctcatgccgatcttccaagagcctgctgcactcaaatggcatcacctacctctattggtacctgcagaagcccgggcaatcccctcagttgctcatctatcagatgtctaaccgcgcctccggtgtccccgaccgcttcagctcctctggctccggcaccgactttactctgaagatatcccgcgtggaggccgaagatgtgggcgtgtactactgcactcagaacctggaactgcccttcaccttcggccagggcaccaagctggagatcaagggcggtggtggctccggcggagggggttctggaggcggcggctcccaggtgcagctggtgcagagcggcgccgaggtgaagaagccgggtgcttccgtgaaggtgtcctgtaaggcctctggctacaccttcaccagctacgtgatgcattgggtccgccaggcccccggacagcgcctggagtggatcggttacatcatcccgtacaacgacggcactaagtacaacgagaaatttcagggccgagtgaccctgacctccgacaaatccagctcgaccgcctacatggagctgtcttctctgcgctcggaggacaccgcggtttattactgtgctcgttggaactgggatggctatttcgacgtgtggggccagggaacgaccgtcaccgtgtcgtccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc